(19) United States
(12) Patent Application Publication
(10) Pub. No.: US 2017/0159134 A1
(43) Pub. Date: Jun. 8, 2017

(54) DNA-Based Method for Forensic Identification of Controlled Substances Using Plant DNA Markers

(71) Applicants: David Knaebel, Manlius, NY (US); Kathleen Corrado, Jamesville, NY (US); Michael Marciano, Manlius, NY (US)

(72) Inventors: David Knaebel, Manlius, NY (US); Kathleen Corrado, Jamesville, NY (US); Michael Marciano, Manlius, NY (US)

(73) Assignee: Syracuse University, Syracuse, NY (US)

(21) Appl. No.: 14/957,834

(22) Filed: Dec. 3, 2015

(51) Int. Cl. C12Q 1/68

(52) U.S. Cl. CPC C12Q 1/6895; C12Q 2600/156; C12Q 2600/16

(57) ABSTRACT

A method for determining the presence or absence of an illicit plant-derived compound in a sample. The method includes the steps of: (i) providing a sample potentially containing an illicit plant-derived compound; (ii) extracting DNA in the illicit plant-derived compound from the identified sample; (iii) amplifying, using PCR, the target plant DNA sequence from the extracted DNA; and (iv) detecting an amplified target plant DNA sequence, where detection of the amplified target plant DNA sequence indicates the presence of the illicit plant-derived compound.

### GOVERNMENT FUNDING

This invention was made with Government support under Grant Number N41756-14-C-3260 awarded by the Department of the Navy, Navy Engineering Logistics Office (NELO). The United States Government has certain rights in the invention.

### FIELD OF THE INVENTION

The present disclosure is directed to the identification of one or more target compounds in a plant isolate, and particularly to the forensic identification of illicit plant-derived target compounds using a novel PCR-based assay. The DNA-based methods can be utilized to replace current qualitative methods of drug identification.

### BACKGROUND

Local and federal law enforcement officers such as police officers, U.S. Customs and Border Protection agents, and forensic investigators often encounter materials that appear to be a controlled substance such as a pharmaceutical compound or an illicit drug. These materials can be found in the field, such as during traffic stops or during questioning or searches, as well as at crime scenes, and can be observed or recognized by the officer, agent, or detective based on appearance, smell, or a variety of other physical characteristics. However, it is necessary to analyze the material using forensic techniques in order to determine with reasonable certainty whether it comprises one or more of the suspected controlled substances. Although rudimentary kits exist to perform an initial test of the material in the field, all confirmatory and/or in-depth testing is performed by a forensic or designated drug identification laboratory.

In the United States for example, an “illicit compound” includes but is not limited to anything defined or regulated by the Controlled Substances Act. The Drug Enforcement Administration, under the auspices of the U.S. Department of Justice, enforces the Controlled Substances Act to suppress the use and distribution of those numerous substances, as well as some of the precursor chemicals used to produce the controlled substances. However, the term “illicit compound” can include chemicals, substances, derivatives, and isolates other than those listed in or regulated by the Controlled Substances Act. For example, an “illicit compound” can be a chemical, substance, derivative, isolate, and/or other compound as defined by a state or other local government. As yet another example, an “illicit compound” can be a chemical, substance, derivative, isolate, and/or other compound as defined by a foreign (non-U.S.) law or international treaty. Many of the same chemicals, substances, derivatives, isolates, and/or compounds will be listed in all of these examples, but others may be listed in only one or a few such examples.

Currently, most powders, pills, liquids, or residues suspected of comprising a target compound undergo an initial color test during forensic analysis. This simple chemical testing—called presumptive testing—provides an initial screen of the compound and can narrow down the additional qualitative and/or quantitative analytical techniques that must also be performed. Examples of color tests include cobalt thiocyanate which turns blue in the presence of cocaine; Dille-Koppanyi reagent which turns light purple or violet-blue in the presence of barbiturates; Marquis reagent which can detect opiates (such as codeine or heroin), phenethylamines (such as mescaline), and alkaloids (such as psilocin, cocaine, caffeine, and nicotine); Zwikker reagent which turns light purple in the presence barbiturates such as phenobarbital; and Froehde reagent which can indicate the presence of opiods; among many, many other such color tests and reagents. In 2000, the National Institute of Justice issued a standard called “Color Test Reagents/Kits for Preliminary Identification of Drugs of Abuse” (NIJ Standard-0604.01) that was developed by the Office of Law Enforcement Standards of the National Institute of Standards and Technology. The standard specifies performance and other requirements that testing equipment (such as color kits) should meet to satisfy the needs of criminal justice agencies for high-quality analysis.

Most often, the material will have to undergo more definitive confirmatory qualitative and quantitative analyses even if target compounds are presumptively identified. Examples of this confirmatory analysis, which typically comprises information about the chemical structure of the material, include gas chromatography (GC), liquid chromatography (LC), mass spectrometry (MS), Fourier transform infrared spectrophotometry (FTIR), and Raman spectroscopy, among other methods.

However, the process of presumptive testing and subsequent confirmatory qualitative analysis suffers from several drawbacks. For example, each of the presumptive testing and subsequent confirmatory analysis can include multiple steps with multiple reagents, vials, or other components. Additionally, specialized instrumentation requiring calibrations, maintenance, and continual replacement parts can be costly. As a result, this multi-step testing is expensive in terms of reagents, equipment, time, and manpower, among other expenses. Further, at least for presumptive testing using color-based kits, there must be an appreciable amount of the suspected compound for a color change to be detected by the naked eye, which is itself a subjective standard.

Accordingly, there is a need in the art for the identification of illicit plant-derived target compounds using a qualitative analytical assay that is affordable, quick, and efficient. The methods described or otherwise envisioned herein may also be utilized for quantitative analysis.

### SUMMARY OF THE INVENTION

The present disclosure is directed to inventive methods for identifying illicit plant-derived compounds in materials suspected of comprising the illicit compound. The inventive method includes identification of the suspect material, extraction of plant DNA from the material, and a PCR-based assay for definitive typing of the plant-derived compound. Several different DNA assays can be used in concert in order to provide a high level of confidence. Further, as a result of the design of the PCR-based assay, several assays can be performed simultaneously to assess the identity of a suspected plant-derived compound. Thus, using the PCR-based assay, the inventive methods can identify illicit plant-derived compounds in materials suspected of comprising the illicit compound in an affordable, quick, and efficient manner.

According to one aspect is a method for determining the presence or absence of an illicit plant-derived compound in a sample, the method including the steps of: (i) providing a sample potentially comprising an illicit plant-derived compound; (ii) extracting DNA in the illicit plant-derived compound from the identified sample; (iii) amplifying, using PCR, a target plant DNA sequence from the extracted DNA; and (iv) detecting an amplified target plant DNA sequence, where detection of the amplified target plant DNA sequence indicates the presence of the illicit plant-derived compound.

According to an embodiment, the illicit plant-derived compound is a plant-derived compound controlled by the Controlled Substances Act.

According to an embodiment, the illicit plant-derived compound is a plant-derived compound controlled by a state government.

According to an embodiment, the method further includes the step of identifying a forward primer and a reverse primer specific to the target plant DNA sequence.

According to an embodiment, the target plant DNA sequence is unique to the genus or species of the plant used as the source of the illicit plant-derived compound.

According to an embodiment, the method further includes the step of purifying the extracted DNA.

According to an embodiment, the step of detecting the amplified target plant DNA sequence comprises qPCR and/or DNA sequencing.

According to an embodiment, the absence of an amplified target plant DNA sequence indicates the absence of the illicit plant-derived compound.

According to an aspect is a method for confirming the presence of an illicit plant-derived compound in a sample. The method includes the steps of: (i) providing a sample, the sample potentially comprising an illicit plant-derived compound; (ii) extracting DNA in the illicit plant-derived compound from the identified sample; (iii) amplifying, using a PCR-based method, a target plant DNA sequence from the extracted DNA, wherein the target plant DNA sequence is unique to the genus or species of the plant used as the source of the illicit plant-derived compound; (iv) detecting an amplified target plant DNA sequence; (v) sequencing at least a portion of the amplified target plant DNA sequence, wherein the sequence confirms the presence of the illicit plant-derived compound in the sample.

These and other aspects of the invention will be apparent from and elucidated with reference to the embodiment(s) described hereinafter.

### BRIEF DESCRIPTION OF THE DRAWINGS

The present invention will be more fully understood and appreciated by reading the following Detailed Description in conjunction with the accompanying drawings, in which like reference characters generally refer to the same elements throughout the different views.

FIG. 1 is a flowchart of a method for the identification of illicit plant-derived target compounds in accordance with an embodiment.

FIG. 2 is a chart of guidelines for the selection of plant DNA sequences sufficient to identify the source of the DNA, in accordance with an embodiment.

### DETAILED DESCRIPTION OF EMBODIMENTS

The present disclosure describes embodiments of inventive methods for identifying plant-derived compounds in sample materials. For example, material that is believed to contain, or could contain, plant tissue or plant-derived compounds such as illicit and/or controlled chemicals (marijuana, cocaine, heroin, etc.) is analyzed using the embodiments described herein. If it is present in the sample, plant DNA from the source of the illicit or controlled chemical is extracted and then a PCR-based assay is utilized to definitively identify the plant-derived compound. As a result of the design of the PCR-based assay, several assays can be performed simultaneously to assess the identity of a suspected plant-derived compound.

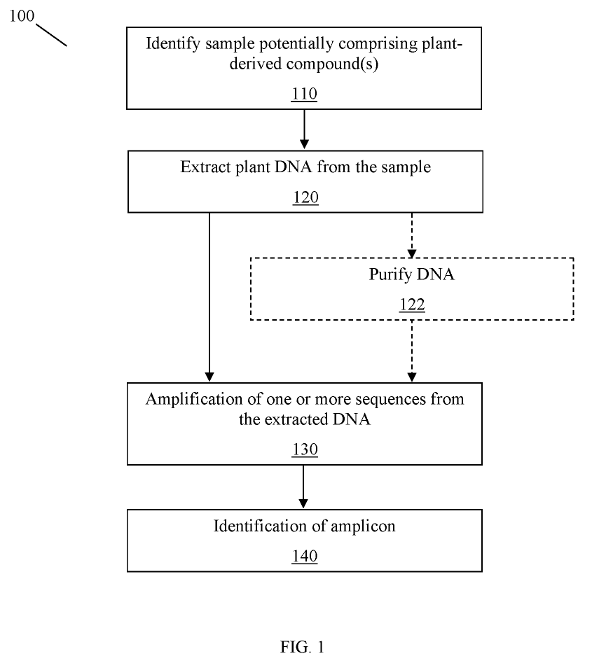

Referring to FIG. 1, in one embodiment, is a flowchart of a method 100 for identifying one or more plant-derived compounds in a sample. At step 110 of the method, a sample is identified and/or collected which will potentially comprise one or more plant-derived compounds. For example, police officers, customs agents, investigators, and forensic scientists often encounter materials that appear to be a controlled substance such as a pharmaceutical compound or an illicit drug. These materials can be found in the field, such as during traffic stops or during questioning or searches, as well as at crime scenes. In addition, samples could be found at sampling or monitoring stations such as airports, bus stations, train stations, or other public transportation locations, among many different types of locations.

According to an embodiment, the sample is any powder, pill, liquid, plant tissue, or residue. As such, the sample may comprise a large amount of the suspect material, or may comprise only a trace amount of the material. As one example, the material is swabbed from the surface of skin, flooring, a car interior, the interior or a fixture of a home or other building, or any of a wide variety of other surfaces which can be swabbed. In the case of swabs, the swab with the collected material may be immediately analyzed or can be stored, and/or can be transported to another location such as a forensic laboratory for subsequent steps of the method. As another example the material is an appreciable amount of plant tissue, powder, or is one or more pills. The collected plant tissue, pills, or powder are then immediately analyzed, stored, and/or transported for analysis. In some instances, the swab, powder, pill, liquid, or residue is processed prior to the next step of the method. For example, a pill can be crushed or otherwise pulverized to create a powder with greater surface area, allowing for improved analysis. A liquid may be allowed to dry before being analyzed.

At step 120 of the method depicted in FIG. 1, the plant DNA—if present in the sample—is extracted from the material. Extracting the plant DNA from the sample will facilitate the PCR-based assay described below. For example, extracting the DNA will remove contaminants and other PCR-inhibiting factors which can result in false negative results. According to an embodiment, the plant DNA is extracted from the sample using standard molecular purification techniques such as silica column based DNA isolation.

According to an embodiment, at optional step 122 of the method, the extracted DNA may be subjected to one or more rounds of purification or modification in order to prepare the DNA for storage, transportation, and/or subsequent analysis. Once the plant DNA is extracted from the sample, well-known purification methods, kits, and systems can be utilized to further prepare the DNA for analysis. Extracted DNA can be analyzed immediately or can be stored for future analysis. For example, the extracted DNA may be sent to another laboratory for presumptive or confirmatory testing. Additionally, the extracted DNA can be divided into separate portions for storage and/or analysis. For example, a portion of the extracted DNA can be stored for future analysis while another portion of the extracted DNA is immediately analyzed via one or more of the subsequent steps of the methods described or otherwise envisioned herein.

At step 130 of the method, a PCR-based assay is utilized to analyze the extracted DNA and identify the plant-based source(s) of that DNA. According to an embodiment, the PCR-based assay amplifies one or more sequences found in the DNA extracted from the sample. Accordingly, the PCR-based assay will comprise one or more unique primer sets utilized to amplify the one or more sequences, as discussed in greater detail below.

According to an embodiment, one or more sequences in the genome of the plant used to generate the plant-based pharmaceutical or drug are identified as targets for the PCR-based assay, and primers are designed to amplify this region. To avoid misidentification, these sequences are designed to be specific to the plant utilized as the source of the drug. For example, often illicit drugs are manufactured from a plant species that has close genetic relatives, including other species, which cannot be used to manufacture the drug. Accordingly, the sequences selected to identify the species used to create the illicit drug must be unique to the target plant species. Although often this will require differentiation between species, sometimes simply identifying a particular genus, family, or order may be sufficient for identification, and thus the specificity of the sequences can be designed accordingly. The primers used to amplify that sequence can also be unique to the species, although in some embodiments the primers can be universal to a family, genus, or other grouping of plants, particularly where the amplified sequence is analyzed to identify the plant DNA in a sample rather than embodiments where the existence or non-existence of an amplicon is used to identify the plant DNA in a sample.

As just one example, the plant Papaver somniferum, known as the opium poppy, is a member of the Papaveraceae family, which includes species such as Papaver rhoeas and Papaver argemone that are not utilized to collect opium. While simply identifying DNA from a sample as belonging to the Papaveraceae family or the Papaver genus may be sufficient, for some assays it might be necessary to differentiate between the P. somniferum, P. rhoeas, and/or P. argemone species. In that case, the genomic sequence identified for amplification must be unique to only P. somniferum and not found in the P. rhoeas or P. argemone species.

### Example—Identifying Target Sequences

According to an embodiment, one or more methods are used to identify a specific sequence in the genome of the plant used to generate the plant-based pharmaceutical or drug. For example, the method described in Example 1 is an example of a process for identifying the specific sequence in the genome of the plant used to generate the plant-based pharmaceutical or drug. The method described in Example 1 is a platform-agnostic method that describes the generation and mining of DNA sequence data. The method could be utilized with, for example, a variety of sequencing platforms, including but not limited to next generation sequencing methods such as Illumina bridge amplification and Roche 454/pyrosequencing.

A plant sample of interest was used to create next generation sequencing libraries, one comprised of pristine genomic DNA and a second comprised of a 4-hour Cot-filtered/duplex-specific nuclease (“DSN”) treated library. The standard sequencing approach for the identification of potential polymorphic sequences in non-model organisms (organisms with little or no sequence data publically available) is shotgun sequencing however amplicon sequencing is also a method that can be used if primer sequences are available. Following the sequencing run, assembly was performed using a de novo assembler program, each of the sequencing runs were both individually assembled and batch assembled. Although assembly is not critical to the identification of simple sequence repeats (“SSRs”) (also known as short tandem repeats (“STRs”) or microsatellites) or single nucleotide polymorphisms (“SNPs”), the assembled data can improve data quality and allow for diagnostic data comparisons.

According to an embodiment the sequence data is organized into several different folders/files, with the most useful containing the raw read file. This file contains the raw sequence data for all sequencing reads that were obtained on a particular sequencing run and is independent of assembly. The average read size range target is 100-400 base pairs which is within the optimal sizes ranges as required by both high resolution melt analysis and capillary electrophoresis and therefore makes the raw reads file the most significant sequencing file for the mining of SSRs and SNPs. A second file with significant utility is a contiguous sequence file which is generated following sequence assembly. This file is a collection of contiguous sequences assembled from overlapping reads files. The assembly data excludes non overlapping reads therefore a significant amount of sequence data may be excluded from downstream analysis. This file has limited utility for variant screening because it excludes unassembled reads. Despite the exclusion of non-overlapping sequence data, the contiguous sequence file can provide a variety of diagnostic data as well as having useful applications for obtaining general measures of quality assurance. The assembled data is most useful when determining the proximity of the variant sequences to one another on a chromosome or within the genome.

Following sequencing, according to an embodiment, the files containing the sequencing reads are mined for microsatellite or SNPs sequences using a software program. For example, one such software program is the Tandem Repeats Finder (TRF) program (https://tandem.bu.edu/trf/trf.html). TRF uses a variety of user-inputted parameters to allow the user to target specific classes of microsatellites. These parameters include, match, mismatch, indel scoring, maximum repeat unit size, and a minimum alignment score. Utilization of a standard set of mining parameters does not allow for significant enough breadth of coverage to target the all the possible classes of microsatellites in the sequence data. Due to the genomic mining of non-model organisms, there is a need to evaluate a spectrum of microsatellite classes from simple repeats of shorter overall length to more complex longer repeats. Accordingly, a sliding parameter range was adopted, where parameters can be adjusted to select for increasing simple repeats or relaxed to obtain more complex repeats.

The targeting of specific classes of microsatellites for the purposes of population or individual discrimination is a complex and highly variable process. The size, sequence of repeat unit, overall sequence complexity, and genomic location (coding vs. noncoding) are examples of conditions that can have substantial impacts on the mutation rates and thus affect the utility of the microsatellite(s) in obtaining the desired level of discrimination. The selected SSRs are then assayed for their ability to be successfully amplified followed by their ability to discriminate between populations, species, and so on depending on the desired level of specificity. Results from an initial batch of SSR data can then be utilized and applied to hone future selection criteria.

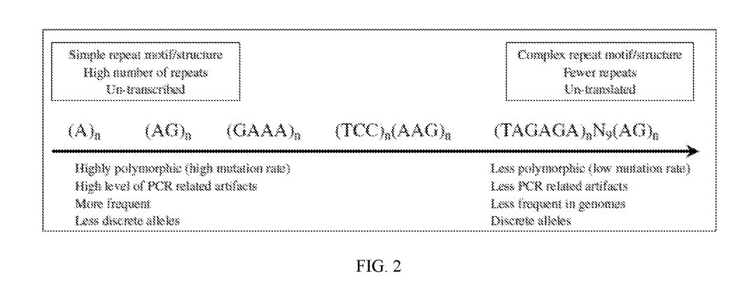

Exemplary guidelines for the selection of suitable sequences are summarized, for example, in FIG. 2, and include the following:
Balance the amount of perfect repeats with the level of interruption. For example, look for strings of three-repeat units that remain intact with interruption;
Strings of mononucleotide repeats can, even when acting as the “interrupters,” lead to higher mutations rates and/or the presence of microvariants;
The optimal number of repeats in an SSR for the PCR-based assay described or otherwise envisioned herein can be an intermediate number and depending on other factors such as number of repeat units and complexity will likely be in the 5-20 range;
Compound SSRs, which have two distinct repeat units that could add to the overall complexity, however the repetitive regions may also have a combined higher mutation rate. Notably, compound repeats separated by a stretch of non-repetitive sequence need to be evaluated individually and together.

In addition to the above general guidelines, the comprehensive characterization of human microsatellite and SNP loci may be an asset when attempting to characterize loci for the target species. The structure, mutation rates, slipped strand mispairing rates, genomic location and general population distribution of microsatellite and SNP loci commonly used in human identification were used as a reference. Human SSR examples illustrate the unpredictable nature of microsatellites and thus support the use of a conservative mining protocol to prevent the exclusion of potentially useful SSRs.

TRF output not only includes the identified microsatellite but also includes the flanking sequences. Accordingly to an embodiment, following the initial screening of the TRF output based on the aforementioned criteria, the flanking sequences were blasted using a locally running BLAST X database. The previously described Cot/DSN protocol allows for the removal of highly repetitive DNAs such as retro elements and chloroplast sequence. The use of Blast X searches of flanking sequences ensures microsatellites or SNPs located within these highly repetitive elements are not selected for further analysis. Alternate methods will be employed for use when ordering primer from sequences which have “hits” to the targets. The use of Blast X on the flanking sequences provides another level of enrichment for the microsatellites or SNPs of interest.

Primers can then be designed using the identified flanking sequences, either by hand or using software. For example, in this example the primers were designed by uploading the identified sequences containing the microsatellites or SNPs to a local version of the Primer 3 software. The primers were then conjugated to fluorophores and used to amplify DNA from the target species and run on a capillary electrophoresis platform. This served as a test for overall primer functionality, identifying the uniqueness of the complements of primer pair throughout the genome (ensuring the PCR target was single copy) and had little non-specific activity. In practice this can be, for example, a qPCR or sequencing based-assay.

According to an embodiment, the PCR-based assay can comprise a multiplex reaction in order to potentially identify more than one type of plant DNA. For example, the assay can comprise a plurality of reactions in one reaction mixture or multiple reaction mixtures. As just one example, a single PCR reaction mixture can comprise the primers for more than just one target sequence. For example, the PCR reaction mixture can include a primer pair for each of the plant species used to make cocaine, heroin, and peyote, among many other combinations and variations. Another example would be a PCR reaction mixture including primer pairs for a family of psychotropic mushrooms, or for all possible sources of psychotropic mushroom extracts. Many other examples are possible. These examples would be especially useful for scenarios where there are different possible sources for the original target, or where a broad spectrum analysis is appropriate. As another example, multiple different analyses can be carried out simultaneously in different reactions. A first reaction would contain a primer pair for a sequence that identifies the coca plant, while a second reaction contains the primer pair to identify the opium plant, while a third reaction contains the primer pair to identify a psychotropic fungi. As yet another example, all possible primer pairs to identify all target psychotropic fungi are used individually in separate, but simultaneous, reactions. Indeed, any different multiplex reactions are possible.

According to an embodiment, the reaction for the PCR-based assay comprises the following conditions. For example, the conditions described below represent a standard “touchdown” PCR protocol used for the evaluation of several primer sets simultaneously. Once specific primer sets are selected, the parameters can be adjusted to the primer optimal annealing temperature. If qPCR is utilized then an intercalating dye or Taqman assay (Thermofisher Waltham, Mass.) method must be used to detect the accumulation of product.

According to another embodiment, the PCR assay comprises the following method, or variations of the following method. Amplification of a template that is known to have very low template concentrations, a high GC content, or complex secondary structure, among other variations, may require more significant modifications of this method. Additionally, the presence of contaminants or PCR inhibitors will often require modifications, some of which might be estimated based on available information about the original sample or the expected plant DNA, although some modifications may have to be experimentally derived.

TABLE 1
PCR Master Mix
1 x Master Mix

| # | Reaction Component | Volume (μL) |
|---|---|---|
| 1 | dH₂O | 10.50 |
| 2 | 10x PCR Buffer + MgCl2 | 1.49 |
| 3 | dNTPs | 0.29 |
| 4 | Roche High Fidelity | 0.11 |
| | Fast Start Taq | |
| 6 | Sample | 2.0 |
| 7 | Primer | 1.05 |

TABLE 2
Sample Thermocycling Program for Touchdown PCR

| | | | | | Cycle |
|---|---|---|---|---|---|
| Cycle Parameter | | Temperature | Duration | | number |
| Denature/Activation | | 95° C. | 5:00-15:00 | min | 1 |
| Phase I | Denature | 95° C. | 0:30 | sec | 10 |
| (touchdown) | Anneal | 65-60 | 0:45 | sec | |
| | | (step 0.5-1° C.) | | | |
| | Extend | 72° C. | 1:00 | min | |
| Phase II | Denature | 95° C. | 0:30 | sec | 20-30 |
| | Anneal | 60° C. | 0:45 | sec | |
| | Extend | 72° C. | 1:00 | min | |

| | | | | |
|---|---|---|---|---|
| Termination | 72° C. | 45:00 | min | 1 |

For one PCR assay according to an embodiment of the invention, the following method might be utilized, in which the following reagents are assembled according to the description set forth in TABLE 3. The final concentration of one or more of the components can be varied. For example, it is common to vary the concentration of Mg++ in a reaction, as well as varying the concentration of the polymerase. In addition to varying the final concentration of one or more of the components, the components themselves may be substituted. For example, many different types of polymerase are possible, among many other variations. According to another embodiment, additional components may be added to the assay to promote the production of amplicons. For example, amplification of problematic targets such as like GC-rich sequences may be improved with additives such as DMSO or formamide, among others.

TABLE 3
Sample PCR Reaction Set-Ups

| | 25 μl | | 50 μl | | |
|---|---|---|---|---|---|
| Element | reaction | | reaction | | Final Concentration |
| 10X Standard Taq | 2.5 | μl | 5 | μl | 1X |
| Reaction Buffer | | | | | |
| 10 mM dNTPs | 0.5 | μl | 1 | μl | 200 μM |
| 10 μM Forward Primer | 0.5 | μl | 1 | μl | 0.2 μM (0.05-1 μM) |
| 10 μM Reverse Primer | 0.5 | μl | 1 | μl | 0.2 μM (0.05-1 μM) |
| Extracted | variable | | variable | | variable |
| Sample DNA | | | | | |
| Taq DNA Polymerase | 0.125 | μl | 0.25 | μl | 1.25 units/50 μl PCR |
| Nuclease-free water | to 25 | μl | to 50 | μl | |

The assembled mixture is gently mixed and, if necessary, aliquoted to reaction tubes. The assembled mixture is transferred to the PCR machine for thermocycling. The thermocycling program for a PCR reaction according to an embodiment of the invention can be the following, or variations of the following. Variations may be made as a result of information about the sample or the DNA, among other factors, or can be made experimentally in order to derive the optimum program.

For a PCR assay according to an embodiment of the invention, the thermocycling program set forth in TABLE 4 can be utilized, although variations of this program are possible based on a wide variety of factors.

TABLE 4
Sample Thermocycling Program

| | | |
|---|---|---|
| Step | Temperature | Time |

| | | | | |
|---|---|---|---|---|
| Initial Denaturation | 95° | C. | 30 | seconds |
| ~30 Cycles | 95° | C. | 15-30 | seconds |
| | 45-68° | C. | 15-60 | seconds |
| | 68° | C. | 1 | minute/kb |
| Final Extension | 68° | C. | 5 | minutes |

| | | | |
|---|---|---|---|
| Hold | 4-10° | C. | indefinitely |

For example, the extension of 1 minute/kb of DNA, the time may be adjusted to be sufficiently long such that the longest possible product can be generated. For example, if the possible amplicons in a sample are 1 kb, 2.5 kb, and 4 kb in length, the extension time is set to at least 4 minutes

This can be automatically determined, calculated, or programmed by entering the primers used into a programming component. The programming component will be linked to a database of primers, and will recognize the length of the possible amplicon based on those primers. The programming component will then provide the minimum extension time needed, or can automatically program the thermocycler using that information.

An initial denaturation of 30 seconds at 95° C. will suffice for most amplicons, although a longer initial denaturation may be required for some templates. During thermocycling, a 15-30 second denaturation at 95° C. will normally suffice. The annealing temperature is typically based on the Tm of the primer pair and is typically 45-68° C.

At step 140 of the method depicted in FIG. 1, the amplicon created in step 130—if one is present—is identified. The term “identify” and its variants can mean anything that results in the amplicon being detected or identified, where for example detection can include simply determining whether an amplicon is created, and identification can include determining the exact sequence of that amplicon.

For example, detection of an amplicon can include the process described above in which one or both of a primer pair is conjugated to fluorophores, and following the thermocycling program the PCR products are run on a capillary electrophoresis platform and the presence or absence of fluorescent amplicons is determined. In addition to detecting the presence of an amplicon, the size of the amplicon can be determined using a DNA ladder or similar mechanism. Real-time PCR (“qPCR”) is an example of a method that can detect the presence and possibly the quantity of a DNA sequence in real time during PCR. For qPCR, the detection of products can be via, for example, non-specific fluorescent dyes that intercalate with double-stranded DNA. Alternatively, the amplicon can be sequenced in order to more specifically analyze the plant-based DNA found in the sample. Sequencing can be accomplished using any of an increasingly wide variety of techniques, processes, and methods.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

What is claimed is:

1. A method for determining the presence or absence of an illicit plant-derived compound in a sample, the method comprising the steps of:
providing a sample, the sample potentially comprising an illicit plant-derived compound;
extracting DNA in the illicit plant-derived compound from the identified sample;
amplifying, using a PCR-based method, a target plant DNA sequence from the extracted DNA; and
detecting an amplified target plant DNA sequence, wherein detection of the amplified target plant DNA sequence indicates the presence of the illicit plant-derived compound.

2. The method of claim 1, wherein the illicit plant-derived compound is a plant-derived compound controlled by the Controlled Substances Act.

3. The method of claim 1, wherein the illicit plant-derived compound is a plant-derived compound controlled by a state government.

4. The method of claim 1, further comprising the step of identifying a forward primer and a reverse primer specific to the target plant DNA sequence.

5. The method of claim 1, wherein the target plant DNA sequence is unique to the genus or species of the plant used as the source of the illicit plant-derived compound.

6. The method of claim 1, further comprising the step of purifying the extracted DNA.

7. The method of claim 1, wherein the step of detecting the amplified target plant DNA sequence comprises qPCR.

8. The method of claim 1, wherein the step of detecting the amplified target plant DNA sequence comprises DNA sequencing.

9. The method of claim 1, wherein the absence of an amplified target plant DNA sequence indicates the absence of the illicit plant-derived compound.

10. A method for confirming the presence of an illicit plant-derived compound in a sample, the method comprising the steps of:
providing a sample, the sample potentially comprising an illicit plant-derived compound;
extracting DNA in the illicit plant-derived compound from the identified sample;
amplifying, using a PCR-based method, a target plant DNA sequence from the extracted DNA, wherein the target plant DNA sequence is unique to the genus or species of the plant used as the source of the illicit plant-derived compound;
detecting an amplified target plant DNA sequence; and
sequencing at least a portion of the amplified target plant DNA sequence, wherein the sequence confirms the presence of the illicit plant-derived compound in the sample.

11. The method of claim 10, further comprising the step of identifying a forward primer and a reverse primer specific to the target plant DNA sequence.

12. The method of claim 10, further comprising the step of purifying the extracted DNA.

13. The method of claim 10, further comprising the step of purifying the amplified target plant DNA sequence.

14. The method of claim 10, wherein the illicit plant-derived compound is a plant-derived compound controlled by the Controlled Substances Act.

15. The method of claim 10, wherein the illicit plant-derived compound is a plant-derived compound controlled by a state government.
